(19) United States
(12) Patent Application Publication
(10) Pub. No.: US 2008/0183084 A1
(43) Pub. Date: Jul. 31, 2008

(54) ELECTRONIC MANOMETER

(76) Inventor: Chao-Man Tseng, Taichung (TW)

Correspondence Address: Chao-Man Tseng, 235 Chung - Ho, Box 8-24, Taipei, 235, omitted

(21) Appl. No.: 12/057,392

(22) Filed: Mar. 28, 2008

Related U.S. Application Data
(62) Division of application No. 11/319,624, filed on Dec. 29, 2005.

(51) Int. Cl. A61B 5/02; G01L 7/18

(52) U.S. Cl. 600/485; 73/747

(57) ABSTRACT

An electronic manometer comprises a manometer body installed with an operating unit, a tying strip, a pressure sensor, and a main control unit; wherein the operating unit sets the main control unit to adjust the pressure of the tying strip; the pressure sensor serves to measure blood pressure or to detect irregular pulses; the operating unit switches the manometer body to operated under at least two different operation modes. The operation modes are blood pressure measuring mode and irregular pulse detection mode. The manometer body has no pump for inflating air or has a pump for inflating air. The tying strip is connected to an inflation unit and air draining unit and the main control unit inflates air into the tying strip through the inflation unit and drains air in the tying strip through the air draining unit. Further, the tying strip is connected to an air releasing unit.

The present invention is a divisional patent application of U.S. patent Ser. No. 11/319,624 which is disclosed by and assigned to the inventor of the present invention. Thus the contents of the U.S. patent Ser. No. 11/319,624 is incorporated into the present invention as a part of the present invention.

The first Official Action for U.S. patent Ser. No. 11/319,624 is a Restriction Requirement where the Examiner has required identification of what the Examiner believes to be patentably distinct species of (i) the manometer body comprising a pump and (ii) the manometer not comprising a pump. The applicant selects the species (i) for continuation of the examination of the U.S. patent Ser. No. 11/319,624; and thus the species (ii) is applied as a new patent applicant.

### FIELD OF THE INVENTION

The present invention relates to manometers, and in particular to an electronic manometer, wherein the manometer body operates under at least two different operation modes. The operation modes are blood pressure measuring mode and irregular pulse detection mode. The defects in measuring irregular pulses in the prior art is improved so that the measure results are precise.

### BACKGROUND OF THE INVENTION

In general, those have heart diseases or blood vessel diseases need to record the blood pressures, especially, diastolic pressures and systolic pressures. Furthermore, the patients of high blood pressures need to record the values of blood pressure so as to determine the timing for taking drugs instead of according to the feeling of the patient. Thereby the precision time control is necessary.

In the prior art manometer, the irregular pulses are measured at the time of measuring blood pressures. If the frequency and amplitude of pulse are unstable, it is considered as irregular pulses.

However in general the time duration for measuring irregular pulses is just about one minute, while it is difficult to have a precise value to determine the frequency of the pulses. The accuracy is low. If the time duration to measure the pulse is prolonged, the accurate can be improved. However the user's wrist will feel uneasy. Thereby the home-use manometer cannot provides a desired result. Although the electrocardiogram can be used to determine the irregular pulses, the cost is high and special knowledge is needed. Therefore, it is unsuitable to be used in home.

### SUMMARY OF THE INVENTION

Accordingly, the primary object of the present invention is to provide an electronic manometer, wherein the manometer body operates under at least two different operation modes. The operation modes are blood pressure measuring mode and irregular pulse detection mode. The defects in measuring irregular pulses in the prior art is improved so that the measure results are precise.

To achieve above objects, the present invention provides an electronic manometer which comprises a manometer body installed with an operating unit 18, a tying strip 11, a pressure sensor 16, and a main control unit 15; wherein the operating unit 18 sets the main control unit 15 to adjust the pressure of the tying strip 11; the pressure sensor 16 serves to measure blood pressure or to detect irregular pulses; the operating unit 18 switches the manometer body 10 to operated under at least two different operation modes. The operation modes are blood pressure measuring mode and irregular pulse detection mode. The manometer body has no pump for inflating air or has a pump for inflating air. The tying strip 11 is connected to an inflation unit 12 and air draining unit 13 and the main control unit 15 inflates air into the tying strip 11 through the inflation unit 12 and drains air in the tying strip 11 through the air draining unit 13. Further, the tying strip 11 is connected to an air releasing unit 14, the main control unit 15 control the air releasing unit 14 to release air in the tying strip 11. If the irregular pulse detection mode is performed after the blood pressure measuring mode, the main control unit 15 will decrease the pressure of the tying strip 11 to be below the diastolic pressures of the user to be measured and the pressure value is retained in a predetermined time duration for detecting irregular pulses; and

If the irregular pulse detection mode is performed with no blood pressure being measured, the main control unit 15 will increase the pressure of the tying strip 11 to detect the diastolic pressures, then the pressure of the tying strip 11 is reduced to be below the diastolic pressures of the user; and the pressure is retained in a predetermined time duration for detecting irregular pulses.

The various objects and advantages of the present invention will be more readily understood from the following detailed description when read in conjunction with the appended drawing.

### BRIEF DESCRIPTION OF THE DRAWINGS

FIG. 1 is a block diagram of the electronic manometer of the present invention.

FIG. 2 shows the flow diagram of the present invention.

### DETAILED DESCRIPTION OF THE INVENTION

In order that those skilled in the art can further understand the present invention, a description will be described in the following in details.

However, these descriptions and the appended drawings are only used to cause those skilled in the art to understand the objects, features, and characteristics of the present invention, but not to be used to confine the scope and spirit of the present invention defined in the appended claims.

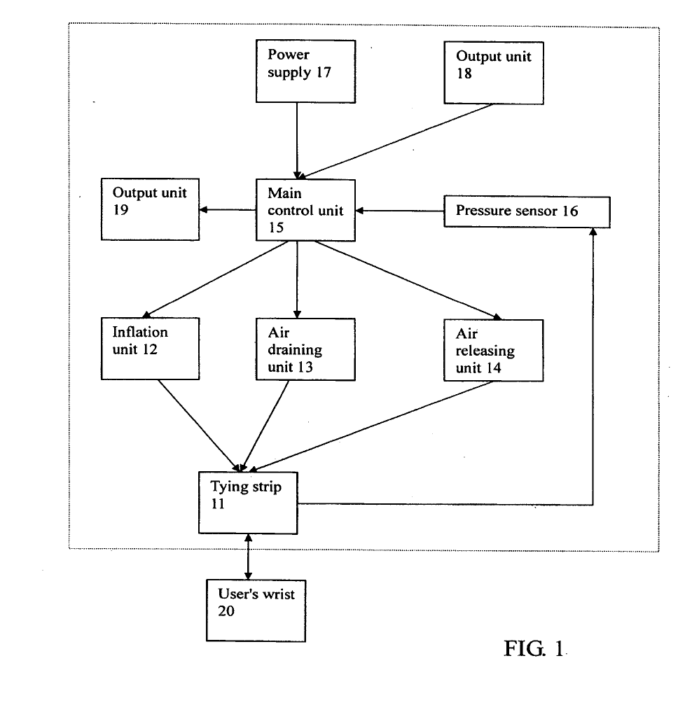

Referring to FIG. 1, the electronic manometer of the present invention is illustrated. The present invention has the following elements.

A manometer body 10 has a tying strip 11 for tying on the user's wrist 20. Air is inflated into the tying strip 11 through an inflation unit 12 and air is drained through an air draining unit 13 for adjusting the air pressure in the tying strip 11. When the measurement of air pressure is complete, the air is released through an air releasing unit 14. Thereby the user's wrist 20 can leave from the tying strip 11.

A main control unit 15 is installed in the main control unit 15 for adjusting the air in the tying strip 11 by controlling the inflation unit 12, air draining unit 13 and air releasing unit 14. The manometer body 10 is connected to a pressure sensor 16 which is installed in the tying strip 11. The pressure sensor 16 serves to sense the pulse of user's heart. The result is transferred to the main control unit 15 from the pressure sensor 16 to be recorded and outputted.

A power supply 17 is installed in the manometer body 10 for supplying power to the whole system. The power supply 17 may be a battery or is the general electric power from the public power system.

An operating unit 18 is installed in the manometer body 10 for adjusting or changing the settings in the main control unit 15.

An output unit 19 is installed in the manometer body 10 for receiving the signals from the main control unit 15 and the output the results which are for example blood pressure or irregular pulses.

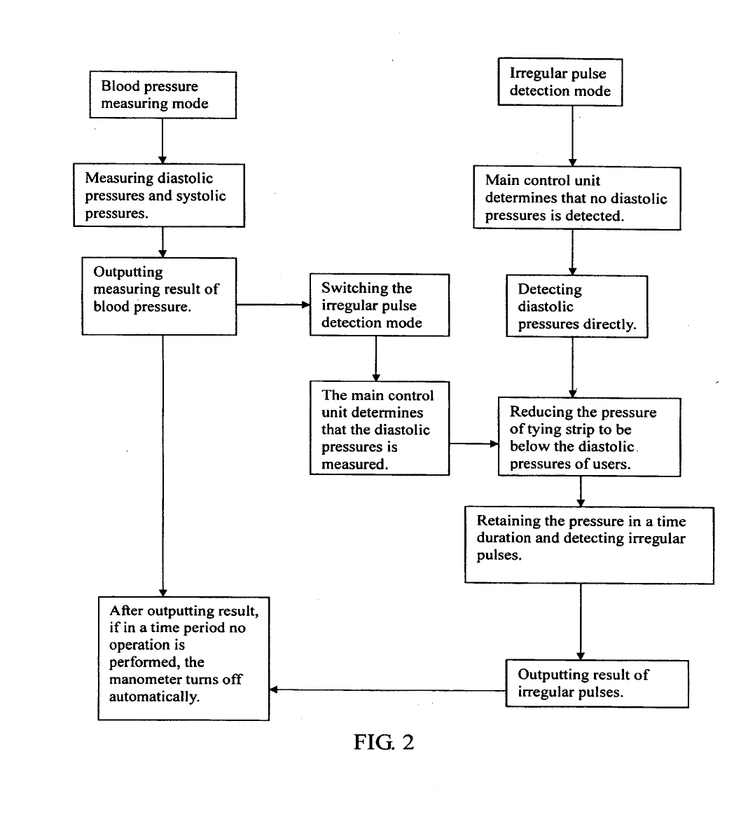

Referring to FIG. 2, it is illustrated that in use of the manometer body 10, the use can select a desired blood pressure measuring mode or irregular pulse detection mode. If the irregular pulse detection mode is selected, the main control unit 15 will determine the unmeasured diastolic pressures. The main control unit 15 operates the inflation unit 12 to inflate air into the tying strip 11 until the tying strip 11 measures the diastolic pressures of those to be measured. Then the systolic pressures is not be measured. The diastolic pressures is used as a base. The air draining unit 13 is operated to release air in the tying strip 11. The pressure in the tying strip 11 is reduced so that the pressure in the tying strip 11 is retained between 100% and 50% of the diastolic pressures of those to be measured. The pressure sensor 16 serves to sense the irregular pulse. The duration of the operation time is set by the operating unit 18 to change the setting in the main control unit 15. The total measuring time is short at this modes so that the result of the irregular pulse detection can be afforded to the use rapidly.

When the blood pressure measuring mode is selected, by the operation of the main control unit 15, the tying strip 11 tied upon the wrist 20 of the user will operate the inflation unit 12 to inflate air into the tying strip 11. The pressure of the tying strip 11 will increased so measure the systolic pressures and diastolic pressures of the user. After measuring the blood pressure, the output unit 19 will output the measure result of the blood pressure to display to the user. If the manometer body 10 is not used, the main control unit 15 is turn off after 90 seconds. The duration for turning off is controlled by the operating unit 18.

If the user is further to perform the irregular pulse detection, the operating unit 18 is switched to the irregular pulse detection mode. At this time, the main control unit 15 uses the diastolic pressures as a base to control the air draining unit 13 to release air from the tying strip 11 so as to reduce the pressure of the tying strip 11. Thereby the pressure value of the tying strip 11 is retained between 100% and 50% of the diastolic pressures of the user. The pressure sensor 16 serves to detect the irregular pulse. The duration for turning off is controlled by the operating unit 18 to change the setting in the main control unit 15. The total measuring time is log and the blood pressure can be measured completely.

After it is used, the main control unit 15 will operate the air releasing unit 14 to release air in the tying strip 11 rapidly so that the user's wrist can release out from the tying strip 11. The result can be outputted from the output unit 19 by sound, characters, figures, vibrations, lights, etc.

In above description, a pump type electronic manometer is used as an example, however the non-pump type electronic manometer is also suitable to be used with the present invention. In the non-pump type electronic manometer, a manual operated compressor device is installed to the tying strip. The compressor device can inflate and release air to the tying strip so as to replace the inflation unit, air releasing unit, and air draining unit.

The present invention is thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. An electronic manometer comprising:
a manometer body installed with an operating unit, a tying strip, a pressure sensor, and a main control unit; wherein the operating unit sets the main control unit to adjust the pressure of the tying strip; the pressure sensor serves to measure blood pressures or to detect irregular pulses; the operating unit switches the manometer body to be operated under at least two different operation modes; and
wherein the manometer body has no pump for inflating air.

2. The electronic manometer as claimed in claim 1, wherein the operation modes are blood pressure measuring mode and irregular pulse detection mode.

3. The electronic manometer as claimed in claim 1, wherein if the irregular pulse detection mode is performed after the blood pressure measuring mode, the main control unit will decrease the pressure of the tying strip to be below the diastolic pressures of the user to be measured and the pressure value is retained in a predetermined time duration for detecting irregular pulses; and
if the irregular pulse detection mode is performed with no blood pressure being measured, the main control unit will increase the pressure of the tying strip to detect the diastolic pressures, then the pressure of the tying strip is reduced to be below the diastolic pressures of the user; and the pressure is retained in a predetermined time duration for detecting irregular pulses.

4. The electronic manometer as claimed in claim 1, wherein the tying strip is connected to the air draining unit and air releasing unit and the main control unit drains air in the tying strip through the air draining unit and releases air in the tying strip through the air releasing unit.

5. The electronic manometer as claimed in claim 2, wherein the tying strip is connected to the air draining unit and air releasing unit and the main control unit drains air in the tying strip through the air draining unit and releases air in the tying strip through the air releasing unit.

6. The electronic manometer as claimed in claim 3, wherein the tying strip is connected to an inflation unit and air draining unit and the main control unit inflates air into the tying strip through the inflation unit and drains air in the tying strip through the air draining unit.

7. The electronic manometer as claimed in claim 1, wherein the tying strip is connected to an air releasing unit, the main control unit controls the air releasing unit to release air in the tying strip.

8. The electronic manometer as claimed in claim 2, wherein the tying strip is connected to an air releasing unit, the main control unit controls the air releasing unit to release air in the tying strip.

9. The electronic manometer as claimed in claim 3, wherein the tying strip is connected to an air releasing unit, the main control unit controls the air releasing unit to release air in the tying strip.

10. The electronic manometer as claimed in claim 1, further comprising an operating unit installed in the manometer body for adjusting or changing the settings in the main control unit and measuring results being outputted to users through the output unit.

11. The electronic manometer as claimed in claim 2, further comprising an operating unit installed in the manometer body for adjusting or changing the settings in the main control unit and measuring results being outputted to users through the output unit.

12. The electronic manometer as claimed in claim 1, wherein the measuring result is outputted from the output unit by sound, characters, figures, vibrations, lights, etc. Claim 19. The electronic manometer as claimed in claim 3, wherein in detecting irregular pulses, the pressures of the tying strip is between 100% and 50% of the diastolic pressures of the user.

13. The electronic manometer as claimed in claim 3, wherein the main control unit changes the detecting time duration of the irregular pulse detection mode.
